(19) United States
(12) Patent Application Publication
(10) Pub. No.: US 2012/0029498 A1
(43) Pub. Date: Feb. 2, 2012

(54) Bipolar Radio Frequency Ablation Instrument

(76) Inventor: Daniel Igor Branovan, Livingston, NJ (US)

(21) Appl. No.: 12/848,036

(22) Filed: Jul. 30, 2010

(51) Int. Cl. A61B 18/18; A61B 18/14

(52) U.S. Cl. 606/33; 606/49; 606/50

(57) ABSTRACT

An electrocautery instrument is disclosed comprising: a handle having a handle axis; a first electrode assembly, the first electrode assembly having a first handle electrode section retained in the handle, a first oblique electrode section extending from the handle, and a first ablation electrode section disposed at an offset distance from the handle axis; and a second electrode assembly, the second electrode assembly having a second handle electrode section retained in the handle, a second oblique electrode section extending from the handle, and a second ablation electrode section disposed at the offset distance from the handle axis, the second electrode assembly being generally congruent to the first electrode assembly, the handle being configured to retain the first ablation electrode section in generally parallel relationship to the second ablation electrode section.

### FIELD OF THE INVENTION

The present invention relates to surgical instruments and, more particularly, to a bipolar radio frequency ablation device for use in the removal of malignant organ tumors.

### BACKGROUND OF THE INVENTION

A common treatment for malignant tumors in human organs, such as nodules on the thyroid or renal masses on the kidney, is surgical removal of most of the respective organ tissue. For example, a thyroidectomy may be performed to deal with malignant thyroid tumors, a procedure which unfortunately results in removal of most of the thyroid tissue. Moreover, undergoing thyroid surgery often poses risks, such as nerve damage or damage to parathyroid glands, and may require that the patient take thyroid hormone supplements following surgery. Alternatives to thyroidectomy are known in the art, including radio frequency (RF) ablation techniques in which the temperature of the target tissue may be raised to a temperature of 50° C. or higher.

For example, Holmer et al. have evaluated ablation methods, as reported in “Bipolar Radiofrequency Ablation for Nodular Thyroid Disease—ex Vivo and in Vivo Evaluation of a Dose-Response Relationship,” J. Surg. Res. 2009 Oct. 29. A study in percutaneous RF ablation for benign thyroid nodules was also described by Kim et al. in “Radiofrequency Ablation of Benign Cold Thyroid Nodules: Initial Clinical Experience,” Thyroid, 2006 April, 16(4):361-7. Kim et al. reported that the ablation electrode used was internally cooled, and that a majority of the patients required conscious sedation when undergoing the ablation procedure.

While there are known in the art RF devices suitable for use in the ablation of liver tumors, for example, most such devices require an extended period of use of from five to ten minutes per session. This length of time makes it impractical to use a conventional RF device on thyroid nodules, in particular, as the thyroid will move with the swallowing motions of the patient. What is needed is an electrocautery or percutaneous ablation instrument that will allow for relatively quick excision of a malignant tissue or tumor.

### BRIEF SUMMARY OF THE INVENTION

In one aspect of the present invention, an electrocautery instrument comprises: a handle having a handle axis; a first electrode assembly, the first electrode assembly having a first handle electrode section retained in the handle, a first oblique electrode section extending from the handle, and a first ablation electrode section disposed at an offset distance from the handle axis; and a second electrode assembly, the second electrode assembly having a second handle electrode section retained in the handle, a second oblique electrode section extending from the handle, and a second ablation electrode section disposed at the offset distance from the handle axis, the second electrode assembly being generally congruent to the first electrode assembly, the handle being configured to retain the first ablation electrode section in generally parallel relationship to the second ablation electrode section.

In another aspect of the present invention, an electrocautery system comprises: a handle having a handle axis; a first electrode assembly partially retained in the handle, the first electrode assembly including a first active electrode distal from the handle; a second electrode assembly partially retained in the handle, the second electrode assembly including a second active electrode distal from the handle, the first active electrode retained in generally parallel relationship with the second active electrode so as to define a bipolar ablation zone therebetween, the bipolar ablation zone being in an offset and substantially parallel alignment with the handle axis; and an RF power supply electrically connected to the first electrode assembly and to the second electrode assembly, the RF power supply functioning to produce a predefined level of ablative RF power in the bipolar ablation zone.

In still another aspect of the present invention, a method for ablating a tissue in a patient comprises the steps of: obtaining an instrument having both a first electrode assembly and a second electrode assembly retained in a handle, the handle retaining the first electrode section in a generally parallel relationship with the second electrode section so as to define a substantially linear bipolar ablation zone between a portion of the first electrode assembly and a portion of the second electrode assembly, the bipolar ablation zone being in an offset and substantially parallel alignment with an axis of the handle; inserting the bipolar ablation zone into a patient proximate a region containing the tissue; determining that a portion of the tissue has been positioned within the bipolar ablation zone; powering the first electrode assembly and the second electrode assembly for a predetermined time period so as to produce a predefined level of ablative RF power in the bipolar ablation zone; and removing the bipolar ablation zone from the patient.

The additional features and advantage of the disclosed invention is set forth in the detailed description which follows, and will be apparent to those skilled in the art from the description or recognized by practicing the invention as described, together with the claims and appended drawings.

### BRIEF DESCRIPTIONS OF THE DRAWINGS

FIG. 1 is an isometric illustration of an electrocautery instrument comprising a handle and a pair of electrode assemblies, in accordance with an aspect of the present invention;

FIG. 2 is a partial-cutaway of the electrocautery instrument of FIG. 1 showing blade contacts, handle electronic sections, and an electronic support insulator secured in the handle;

FIG. 3 is an enlarged view of an ablation electrode section in the electrocautery instrument of FIG. 1;

FIG. 4 is a flow diagram illustrating operation of the electrocautery instrument of FIG. 1;

FIG. 5 is diagrammatical illustration of an ablating system utilizing the electrocautery instrument of FIG. 1;

FIG. 6 is a side view diagram of an exemplary embodiment of an electrocautery instrument, in accordance with the prior art; and

FIG. 7 is a top view diagrammatical illustration of the electrocautery instrument of FIG. 6.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention comprises a bipolar radio frequency (RF) ablation or electrocautery instrument designed for percutaneous ablation of tissue in a human cavity, such as thyroid nodules or renal masses. The instrument may be inserted through a patient's skin to thyroid nodules or to renal cell carcinomas under ultrasound guidance. Activation of the instrument serves to quickly destroy the malignant tissue. The bipolar configuration provides for the ability to localize the region of ablation and to thus minimize peripheral damage to surrounding, healthy tissue.

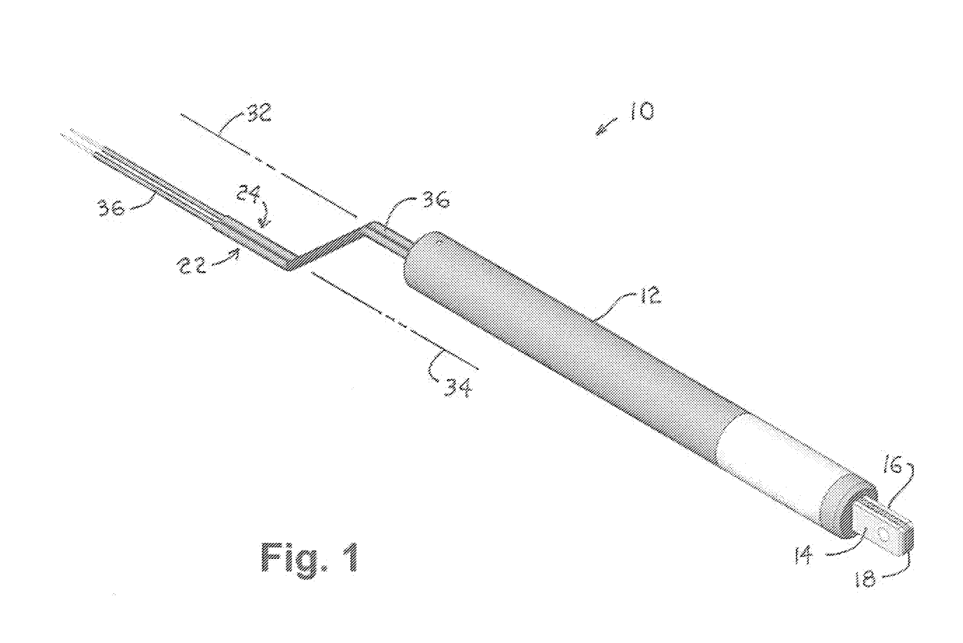

There is shown in FIG. 1 an exemplary embodiment of an electrocautery instrument 10 comprising a handle 12 retaining a first electrode assembly 22 and a second electrode assembly 24. The handle 12 may be fabricated from a nonconductive material, such as a plastic or dielectric. The first electrode assembly 22 and the second electrode assembly 24 are electrically connected to a first blade contact 14 and a second blade contact 16, respectively.

A blade insulation spacer 18 may be disposed between the first electrode assembly 22 and the second electrode assembly 24 so as to electrically isolate the first electrode assembly 22 from the second electrode assembly 24. The first electrode assembly 22 and the second electrode assembly 24 may thus be powered by applying RF power to the first blade contact 14 and the second blade contact 16.

As shown in the diagram, the portions of the first electrode assembly 22 and the second electrode assembly 24 distal from the handle 12 are in an offset configuration. These distal electrode assembly portions lie along an ablator axis 34 that is offset from a handle axis 32 that lies along a centerline of the handle 12. As can be appreciated by one skilled in the art, the offset configuration shown is particularly advantageous providing a clear view of the skin puncture site before insertion of the distal electrode assembly portions into the patient.

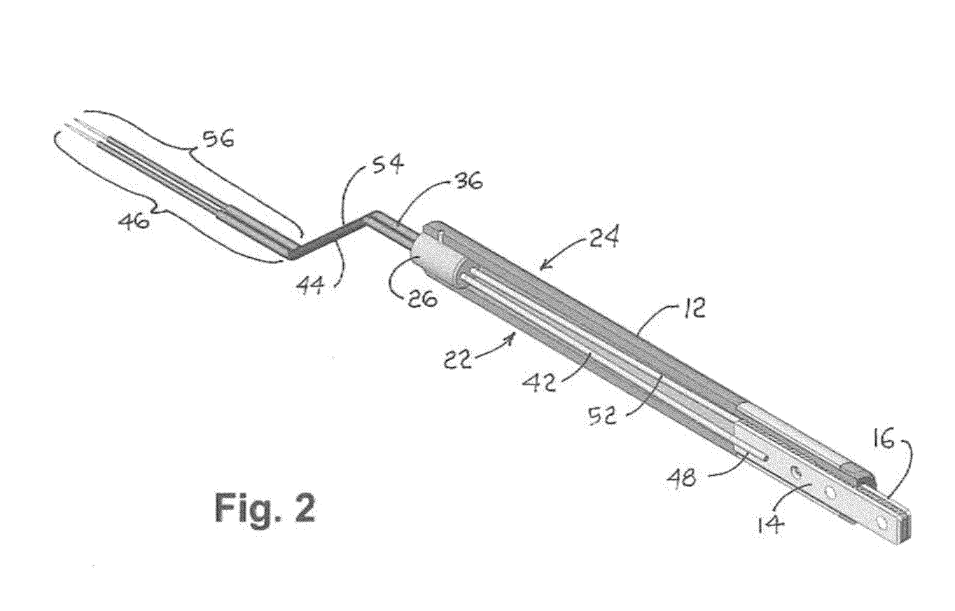

As shown in FIG. 2, the first electrode assembly 22 comprises a first handle electrode section 42, a first oblique electrode section 44, and a first ablation electrode section 46. The first handle electrode section 42 may be electrically connected to the first blade contact 14 at an electrical attachment 48, such as by brazing or welding. The second electrode assembly 24 is similar in configuration to the first electrode assembly 22. Accordingly, the second electrode assembly comprises a second handle electrode section 52, a second oblique electrode section 54, and a second ablation electrode section 56.

The handle 12 is configured to retain the first blade contact 14 and the second blade contact 16 at the rear of the handle 12. An electrode support insulator 26 may be provided at the front of the handle 12 to retain the first handle electrode section 42 and the second handle electrode section 52 in a spaced apart, substantially parallel relationship.

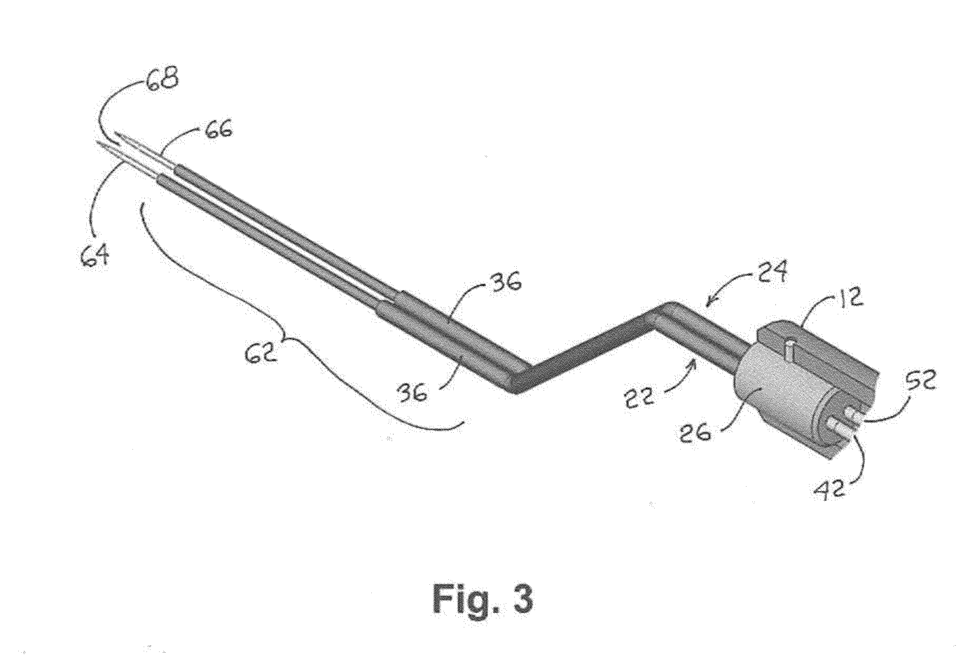

As best shown in FIG. 3, the first ablation electrode section 46 may be partially covered with the insulating sleeve 36 to form an insulated ablation electrode 62 for part of the length of the first ablation electrode section 46, and a first active electrode 64 without the insulating sleeve 36 for the remaining length of the first ablation electrode section 46. Similarly, the second ablation electrode section 56 may be partially covered with the insulating sleeve 36 to form a second active electrode 66, where a bipolar ablation zone 68 may be defined as the region between the first active electrode 64 and the second active electrode 66. This configuration serves to restrict any electrical discharge between the first electrode assembly 22 and the second electrode assembly 24 to the bipolar ablation zone 68.

It can be appreciated that the exposed lengths of the active electrodes 64, 66 determine the size of the resulting ablated lesion. The exposed lengths of the active electrodes 64, 66 are thus a function of the size of the target tumor. In an exemplary embodiment, the spacing between the first active electrode 64 and the second active electrode 66 is specified so as to be able to enclose a thyroid nodule or a renal carcinoma between the first active electrode 64 and the second active electrode 66 for cauterization by the electrocautery instrument 10.

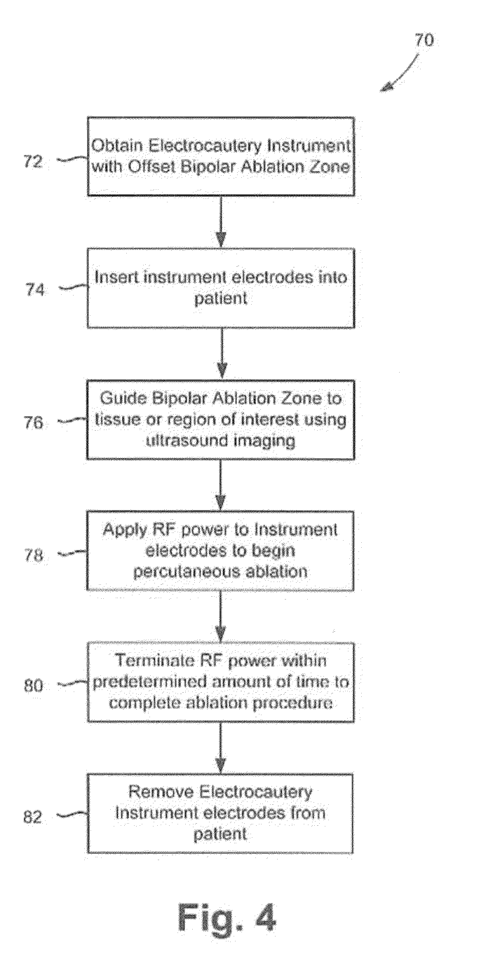

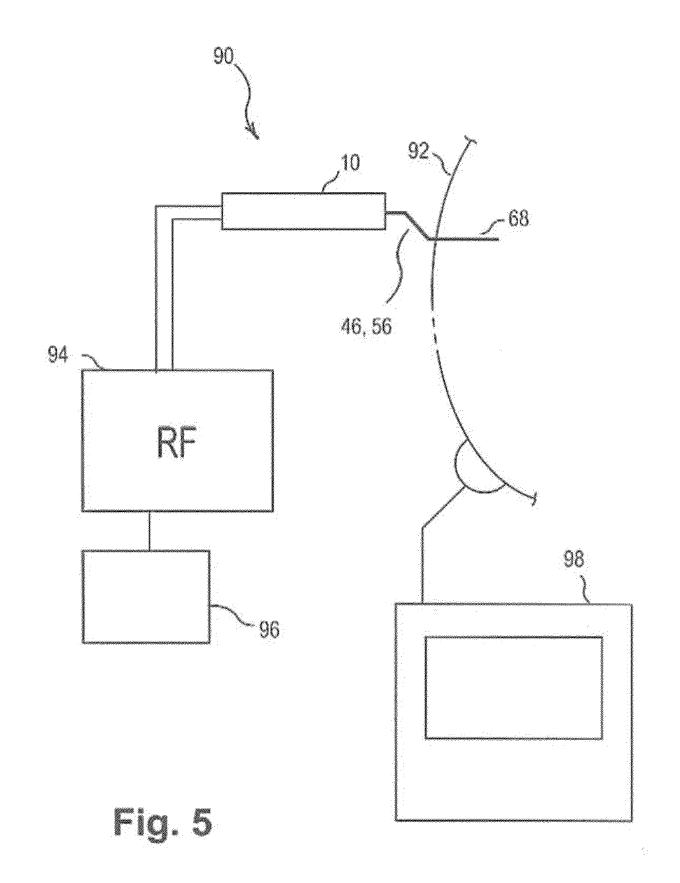

Operation of the electrocautery instrument 10 may be described with reference to a flow diagram 70, shown in FIG. 4, in which the electrocautery instrument 10 with the offset bipolar ablation zone 68 is obtained, at step 72. With additional reference to FIG. 5, the first ablation electrode section 46 and the second ablation electrode section 56 are inserted into a patient 92, at step 74. The bipolar ablation zone 68 may be guided to a target tissue or to a region of interest, such as a thyroid or a kidney, using feedback from an ultrasound imaging unit 98, at step 76.

It can be appreciated that, as the first ablation electrode section 46 and the second ablation electrode section 56 are formed from metal, the location of the first ablation electrode section 46 and the second ablation electrode section 56 inside the patient can be established by means of ultrasound imaging. Power may be applied to the electrocautery instrument 10, at step 78, using an RF power source 94 and control unit 96. In an exemplary embodiment, the RF power source 94 may output between about ten watts and twenty watts of RF power at an operating frequency of about 800 MHz to about 6.0 GHz.

The RF power source 94 may provide ablative energy to the bipolar ablation zone 68 for a predetermined period of time to complete the electrocautery or percutaneous ablation procedure, at step 80. In an exemplary embodiment, the predetermined period of time may comprise a duration of from about ten seconds to about thirty seconds. Because the electrocautery procedure can be completed within the upper time period of thirty seconds, it may not be necessary to have the patient placed under general anesthesia. The control unit 96 may be used to power down the RF power source 94 to terminate the ablation process. The first ablation electrode section 46 and the second ablation electrode section 56 may then be removed from the patient 92, at step 82.

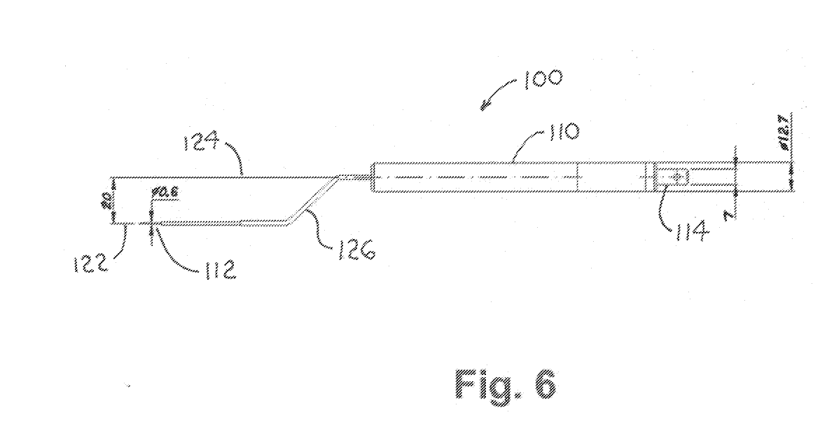

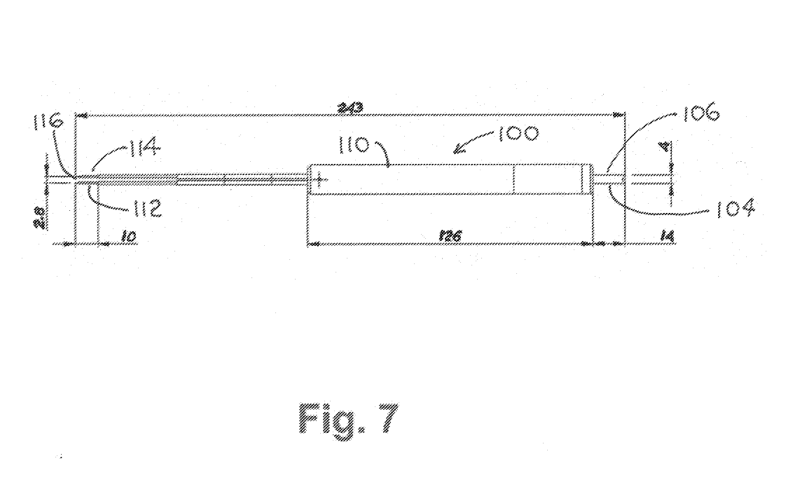

In an exemplary embodiment, an electrocautery instrument 100 may be fabricated as a device having an overall length of approximately 243 mm, as shown in FIGS. 6 and 7. The electrocautery instrument 100 may comprise a handle 110 approximately 126 mm in length and about 12.7 mm in diameter. A first blade contact 104 and a second blade contact 106 are configured to interface with standard RF power supplies and, accordingly, may each have a width of about 7.0 mm, protrude approximately 14 mm from the handle 110, and have outer surfaces spaced at a distance of about 4 mm.

The electrocautery instrument may comprise a first active electrode 112 and a second active electrode 114, each about 10 mm in length. The first active electrode 112 may be spaced from the second active electrode 114 by a distance of about 2.8 mm, although an alternative spacing of from about 2.2 mm to about 3.2 mm would lie within the scope of the present invention. This range of dimensions enables an optimal bipolar cautery to provide for a relatively quick ablation procedure. In addition, damage to surrounding tissue may be mitigated or eliminated by using the relatively quick procedure.

The diameters of the first active electrode 112 and the second active electrode 114 may be about 0.6 mm in diameter. The configuration shown provides for a bipolar ablation zone 116 of about 10 mm by about 2.2 mm. An ablator axis 122 may be offset from a handle axis 124 by a distance of about 20 mm as shown, although an alternative offset distance of from about 10 mm to about 30 mm would also lie within the scope of the present invention. An oblique electrode section 126 may form an angle of approximately 45° with the handle axis.

It is to be understood that the description herein is exemplary of the invention only and is intended to provide an overview for the understanding of the nature and character of the invention as it is defined by the claims. The accompanying drawings are included to provide a further understanding of various features and embodiments of the method and apparatus of the invention which, together with their description serve to explain the principles and operation of the invention. Thus, while the invention has been described with reference to particular embodiments, it will be understood that the present invention is by no means limited to the particular constructions and methods herein disclosed and/or shown in the drawings, but also comprises any modifications or equivalents within the scope of the claims.

What is claimed is:

1. An electrocautery instrument suitable for selectively ablating tissue, said instrument comprising:
a handle having a handle axis;
a first electrode assembly, said first electrode assembly having a first handle electrode section retained in said handle, a first oblique electrode section extending from said handle, and a first ablation electrode section disposed at an offset distance from said handle axis; and
a second electrode assembly, said second electrode assembly having a second handle electrode section retained in said handle, a second oblique electrode section extending from said handle, and a second ablation electrode section disposed at said offset distance from said handle axis, said second electrode assembly being generally congruent to said first electrode assembly, said handle being configured to retain said first ablation electrode section in generally parallel relationship to said second ablation electrode section.

2. The instrument of claim 1 wherein said offset distance is in the range of approximately 10 mm to approximately 30 mm.

3. The instrument of claim 1 wherein said first ablation electrode section includes a first active electrode and said second ablation electrode section includes a second active electrode, said first active electrode spaced apart from said second active electrode so as to define a bipolar ablation zone therebetween.

4. The instrument of claim 3 wherein said active electrode spacing is in the range of approximately 2.2 mm to approximately 3.2 mm.

5. The instrument of claim 3 wherein said active electrode spacing is specified for enclosing at least one of a thyroid nodule and a renal carcinoma.

6. The instrument of claim 1 wherein said first electrode assembly further includes an insulation sleeve covering a portion of said first ablation active electrode section.

7. The instrument of claim 1 wherein said first oblique electrode section forms an angle of approximately 45° with said handle axis.

8. An electrocautery system suitable for performing selective ablation of a tissue, said system comprising:
a handle having a handle axis;
a first electrode assembly partially retained in said handle, said first electrode assembly including a first active electrode distal from said handle;
a second electrode assembly partially retained in said handle, said second electrode assembly including a second active electrode distal from said handle, said first active electrode retained in generally parallel relationship with said second active electrode so as to define a bipolar ablation zone therebetween, said bipolar ablation zone being in an offset and substantially parallel alignment with said handle axis; and
a RF power supply electrically connected to said first electrode assembly and to said second electrode assembly, said RF power supply functioning to produce a predefined level of ablative RF power in said bipolar ablation zone.

9. The system of claim 8 wherein said first electrode assembly comprises a first oblique electrode section disposed between said handle and said first active electrode, said first oblique electrode section forming an angle of approximately 45° with said handle axis.

10. The system of claim 8 wherein said first active electrode is spaced from said second active electrode by a distance of from approximately 2.2 mm to approximately 3.2 mm.

11. The system of claim 8 wherein said predefined ablative RF power level in said workspace comprises an output of between approximately ten watts and approximately twenty watts.

12. The system of claim 8 wherein said predefined ablative RF power level functions at an operating frequency lying in the range of approximately 800 MHz to approximately 6.0 GHz.

13. A method for ablating a tissue in a patient, said method comprising the steps of:
obtaining an instrument having both a first electrode assembly and a second electrode assembly retained in a handle, said handle retaining said first electrode section in a generally parallel relationship with said second electrode section so as to define a substantially linear bipolar ablation zone between a portion of said first electrode assembly and a portion of said second electrode assembly, said bipolar ablation zone being in an offset and substantially parallel alignment with an axis of said handle;
inserting said bipolar ablation zone into a patient proximate a region containing the tissue;
determining that a portion of the tissue has been positioned within said bipolar ablation zone;
powering said first electrode assembly and said second electrode assembly for a predetermined time period so as to produce a predefined level of ablative RF power in said bipolar ablation zone; and
removing said bipolar ablation zone from the patient.

14. The method of claim 13 wherein said step of powering is completed within a period of approximately thirty seconds.

15. The method of claim 13 wherein said step of determining comprises a step of guiding said bipolar ablation zone to said tissue using ultrasound imagery.

16. The method of claim 13 wherein said step of determining comprises a step of placing one of a thyroid nodule or a renal carcinoma within said bipolar ablation zone.
